(19) United States
(12) Patent Application Publication
(10) Pub. No.: US 2011/0034823 A1
(43) Pub. Date: Feb. 10, 2011

(54) Lumen diameter and stent apposition sensing

(76) Inventors: Daniel Gelbart, Vancouver (CA); Lindsay S. Machan, Vancouver (CA)

Correspondence Address: DANIEL GELBART, 4706 DRUMMOND DR, VANCOUVER, BC, V6T-1B4, CA

(21) Appl. No.: 12/807,693

(22) Filed: Sep. 13, 2010

Related U.S. Application Data
(62) Division of application No. 12/216,649, filed on Jul. 9, 2008.

(51) Int. Cl. A61B 5/05; A61F 2/82

(52) U.S. Cl. 600/547; 623/1.15

(57) ABSTRACT

A stent balloon is provided with two conductive rings, created by a thin metallized coating deposited directly on the balloon, adjacent to the ends of the stent. The impedance between those rings and the body of the patient is measured at different AC frequencies. As the balloon approaches the vessel wall the impedance increases rapidly. Once the balloon forms full contact with vessel wall the impedance increases slowly. The changing impedance provides a guide for optimal apposition of the stent.

The same conductive rings can also detect stent slippage and stent position relative to the balloon. With the addition of an extra conductive pad and wire, stent spring-back can be measured and corrected for.

### FIELD OF THE INVENTION

The invention is in the medical field and in particular in the field of stenting.

### BACKGROUND OF THE INVENTION

The art of keeping bodily lumens open by using stents is well known and used not only in the vascular system but also for other lumens in the body, such as in the digestive and renal system. In general two conditions need to be met when a stent is deployed: the ends have to have full contact with the lumen along their circumference and the central section has to be sufficiently open. In an ideal stent apposition the ends form a smooth transition to the vessel wall. Both under expansion and over expansion are undesirable, causing increased stenosis and other well known negative effects. The most common use of stents is in the arterial system. The stenting is performed under x-ray (fluoroscopy). The current x-ray tools are not sufficient to judge the apposition because of at least three reasons: lack of resolution, the fact that vessel wall is visible only for a short time when a dye is injected and the fact that the current x-ray system only provides a view from a single viewing angle. When a stent is not fully deployed, for example when it is opened to an oval instead of a round cross section, the diameter seen will depend on the viewing angle. This is illustrated in FIG. 1 where a stent 1 having an oval cross section appears as having a width 5A when viewed from direction A, and a different width 5B when viewed from direction B. Additional problems encountered in stent deployment are stent spring-back and stent slippage relative to the balloon. Stent spring-back is caused by the elasticity of the stent material, making the stent shrink slightly when the balloon is de-pressurized. This problem is mainly found in the Co—Cr stents. The amount of spring-back can not be fully predicted (and compensated for) as the elasticity of the vessel adds to the elasticity of the stent. Stent longitudinal slippage relative to balloon is mainly a problem with stents that are crimped on before use at the hospital, as the crimping is less controlled than the crimping and bonding done at the factory.

Prior art system attempted to sense contact between the stent ends and vessel wall by using pressure sensors. For example, U.S. Pat. No. 6,179,858 uses expansion or pressure sensors based on variable capacitors at the ends of the balloon adjacent to the ends of the stents. Such sensors increase the diameter and complexity of the balloon, as the capacitor is formed between two conductors separated by a dielectric. Such a structure adds at least three layers to the balloon. Modern stents can be deployed in very narrow vessels (below 2 mm). The small size does not allow for any device that may significantly increase the diameter of the balloon in the collapsed state. The sensors of the '858 patent add significant thickness and complexity to the collapsed balloon which has to be as small as 1 mm for some applications. A different approach is disclosed in European patent WO 02/058549. A complex impedance sensing device is built into the stent. Again, since the design is based on an electronic integrated circuit built into the stent it is not suitable to small diameter stents. The prior art also greatly increases the cost of the stents. Another problem with prior art impedance measurement is that the actual impedance of the vessel wall is unknown, as the wall can be clean or covered by various types of plaque. The current invention does not rely on the absolute impedance of the vessel wall. Prior art attempts to sense longitudinal slippage, such as U.S. Pat. No. 6,091,980 required two additional conductors brought out of the patient.

It is an object of the invention to sense the apposition of the stent in a simple manner which has minimal effect on the diameter of the stent or the balloon. Another object is to provide a low cost solution, compatible with current stent balloon construction methods. Still another object is to add, when desired, simple means for detecting stent spring back and stent slippage and to achieve slippage sensing without adding any electrical wires. Other objects and advantages will become apparent when studying the drawings with the disclosure.

### SUMMARY OF THE INVENTION

A stent balloon is provided with two conductive rings, created by a thin metallized coating deposited directly on the balloon, adjacent to the ends of the stent. The impedance between those rings and the body of the patient is measured at different AC frequencies. As the balloon approaches the vessel wall the impedance increases rapidly. Once the balloon forms full contact with vessel wall the impedance increases slowly. The changing impedance provides a guide for optimal apposition of the stent.

The same conductive rings can also detect stent slippage and stent position relative to the balloon. With the addition of an extra conductive pad and wire, stent spring-back can be measured and corrected for.

### BRIEF DESCRIPTION OF THE DRAWINGS

FIG. 1 is a perspective view of prior art stent deployment.

FIG. 2 is a perspective view of a stent balloon incorporating sensing electrodes.

FIG. 3 is a longitudinal section of stent deployment according to the invention.

FIG. 4 is a cross section of the lead wires and tubes connected to the stent balloon.

FIG. 5 is a schematic diagram of an electronic circuit for measuring impedance between the sensing electrodes and the body.

FIG. 6 is a graph of the impedance between the sensing electrodes and the body, measured at different frequencies.

FIG. 7 is a graph of the impedance between the sensing electrodes and the body, measured at different positions of the balloon relative to a deployed stent.

FIG. 8 is a perspective view of a stent balloon also incorporating stent spring-back sensing.

### DETAILED DISCLOSURE

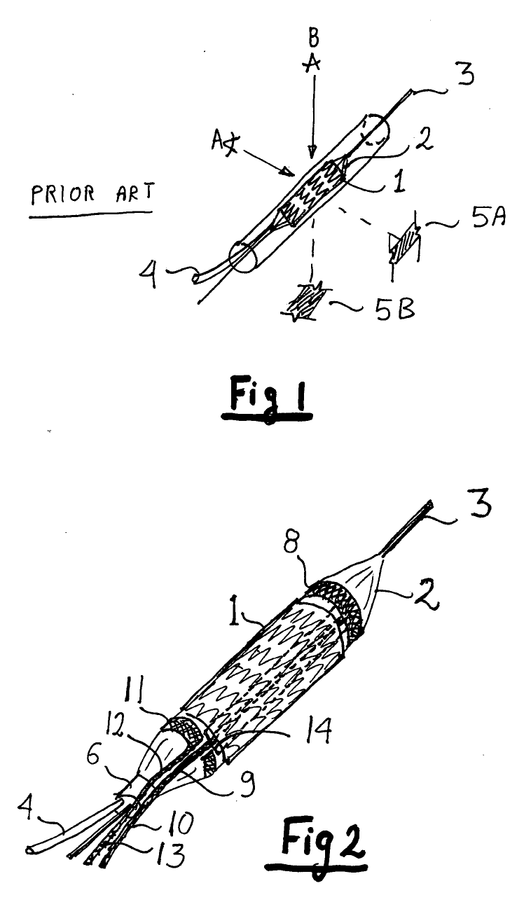

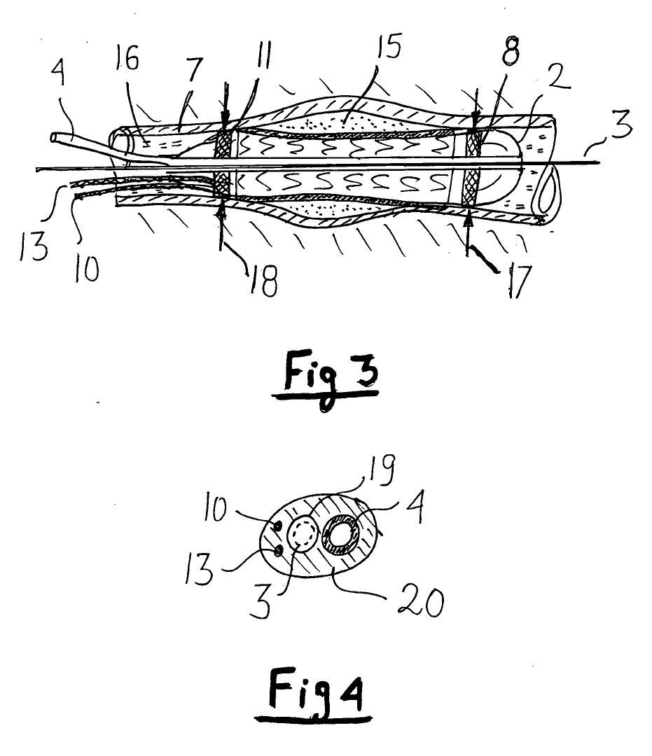

Referring now to FIG. 2, a stent 1 is expanded by balloon 2 connected to a pressurizing tube 4 and guided by guide wire 3. As the art of stents and stenting is well known, no further details are given. The balloon has a distal ring electrode 8 and a proximal ring electrode 11 preferably formed by metallizing the pattern directly onto the balloon. The art of metallizing polymers is well known and used extensively is packaging materials. It can be done by vacuum evaporation, sputtering or chemical deposition. The advantage of metallization is that significant conductivity can be achieved without increasing the diameter of the balloon and without affecting its mechanical properties. A typical thickness of a metallized layer is below 1 micron and can be as thin as 0.1 um. Many metals are suitable for the metallized electrodes, such as aluminum, gold, or nickel. The ductility of the coating can be increased by using a serpentine-like pattern but even a solid area will withstand the expansion of the balloon, as the expansion of the balloon relies on unfolding rather than material stretching. As an alternative a serpentine like wire can be bonded to the balloon and continued to the outside of the body. Typical width of the electrodes is 0.5-2 mm, but width as low as 0.1 mm can still achieve accurate sensing. The electrodes should be placed as close as possible to the stent but without touching it. The electrodes are connected to the end of the balloon via metallized traces 9 and 12, from which very thin conductors 10 and 13 connect them to the sensing unit (not shown). Wires 10 and 13 can be very thin, typically 50 um to 150 um or can also be ribbon shaped. The connection between wires 10, 13 and traces 9, 12 can be preferable done by a polymeric crimping ferrule 6 or by electrically conductive adhesive. In order to avoid electrical contact between the stent 1 and trace 9, a thin insulating coat 14 has to be applied over trace 9 in the area covered by the stent. Such a coat can be made by a thin varnish with good mechanical properties, such as Glyptal, epoxy or polyimide, or by laminating a very thin (2-10 micron) overlay similar to the practice in making flexible printed circuit boards. An alternative is to run wire 10 inside the balloon. Ring electrode 11 has a small gap to allow traces 9 and 12 to pass.

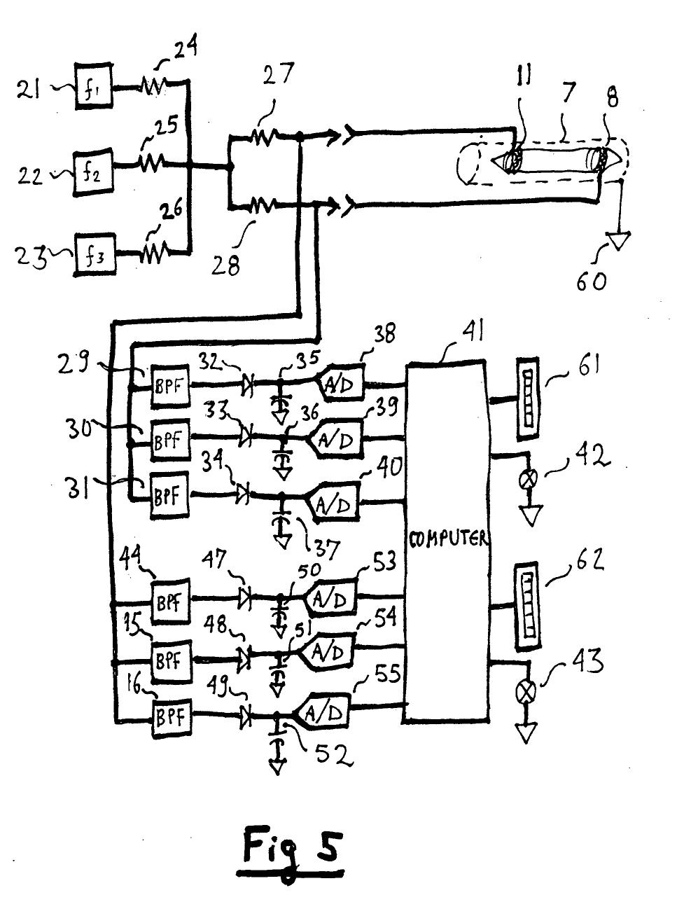

FIG. 3 is a longitudinal section of the stent being deployed. A lumen 7, such as an artery, has a defect 15, such as plaque buildup. A stent 1 is being expanded by balloon 2 to restore flow via lumen 1. For best results the diameter at the proximal end of the stent has to match the lumen diameter 18 and the distal end of the stent has to match the distal diameter 17. Even when the lumen is not round, full contact should be achieved by the circumference of the stent ends and the lumen. When the body of the patient is electrically grounded, either by a grounding pad or by grounding the guide wire 3, the impedance between electrodes 8, 11 and the body will be a function of the tissue in contact with the electrodes. In general blood is more conductive than other tissues such as vessel wall. The conductivity of blood and tissue is a complex subject mainly because of the interface between the tissue and the electrode. For a fuller understanding of tissue impedance a textbook such as “Bioimpedance and Bioelectricity” by Grimnes and Martinsen (ISBN 0-12-303260-1) should be consulted. The measurement should be done using alternating currents, to avoid polarization effects, and can be done at multiple frequencies, for best results. If measurement is done at a single frequency, it should preferably be done in the range of 10 KHz to 1 MHz. As electrodes 8 and 11 approach the wall of vessel 7 the impedance increases as shown in FIG. 6. Once an electrode makes full contact with the vessel wall along the full circumference the impedance increases slowly with further pressure. When the rate of impedance increase slows down the electrode, and stent, is in full contact with the wall. It may be desired to expand the stent slightly more, to allow for secondary factors such as stent thickness, or slightly less, to allow for the balloon bulging beyond the stent. In some cases one end will reach correct apposition before the other, and a trade-off needs to be made by the cardiologist. The extra two electrical leads required for connecting the electrodes to the sensing unit can be incorporated in the pressurizing tube/guide tube assembly currently used. This is shown in FIG. 4. Prior art stents have a pressurizing tube 4, typically made of stainless steel, and a guide tube 19 for accommodating guide wire 3, held together as one unit by polymeric assembly 20. In general the guide wire tube does not extend the full length of the pressurizing tube. Wires 10 and 13 can be molded into assembly 20 and terminated with an electrical connector at the proximal end.

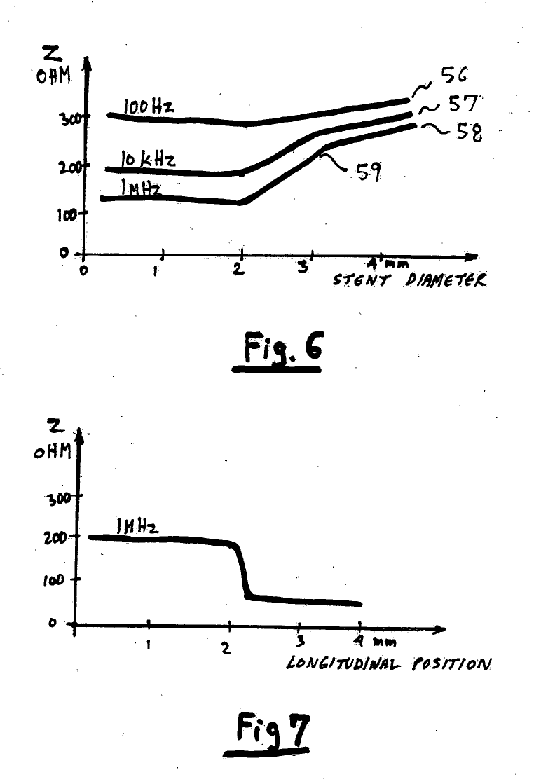

A typical electrical circuit needed for the discrimination between blood and vessel wall is shown in FIG. 5. While the example shows three different frequencies used, any number of frequencies from a single frequency to a continuous frequency sweep can be used. Oscillators 21, 22, 23, having, by the way of example, frequencies of 100 Hz, 10 KHz and 1 MHz, are combined by resistors 24, 25 and 26. Resistors 27, 28 supply electrodes 11 and 8 with the sum of frequencies. The body of the patient is grounded by ground connection 60 and the impedance between electrodes 8, 11 and the body is a complex impedance having a resistive and capacitive component. By using multiple frequencies, not only the impedance but the dispersion of the permittivity can also be measured, for a more accurate discrimination. All three parameter (resistance, capacitance and dispersion) are different between blood and other tissues. Since the vessel wall can be covered with plaque, the exact impedance is less important than the rate of impedance change as stent is deployed, and in particular the point where the change in impedance slows down. The voltage dividers formed by resistors 27, 28 and the impedances to the grounded body formed by electrodes 8 and 11 are used to estimate the position of electrodes 8 and11 relative to vessel wall. These voltages are filtered by band-pass filters 29, 30, 31 for electrode 8 and 44, 45 and 45 for electrode 11. The center frequencies of these filters match the frequencies of the oscillators. The filters can be passive, active or DSP based. The filtered signal is detected by detectors 32, 33, 34 and 47, 48, 49 and filtered by capacitors 35, 36, 37 and 50, 51, 52. A/D converters 38, 39, 40 and 53, 54, 55 couple the signals to a computer 41. The computer displays the approximate distance to the vessel wall on readouts 61 and 62 (for distal and proximal ends) and can include visual and audible warning signals such as lights 42 and 42 when full contact with the vessel wall was achieved (based on the measured impedance and rate of change of the impedance). It will be obvious to those skilled in the art that further refinements are possible, such as having computer 41 automatically control the balloon inflation pump or dividing the balloon into a distal section and a proximal section, each one with its separate pressurizing tube. The latter improvement allows perfect apposition at each end in cases where the distal and proximal vessel diameters are different.

FIG. 6 shows impedance measurements taken in a pig's artery using the invention. The measurements were done using an Agilent (HP) model 3577A Network Analyzer. Resistors 27, 28 in FIG. 5 were 220 Ohms in order to be matched to the typical impedances measured. Graphs 56, 57 and 58 show the impedance change using frequencies of 100 Hz, 10 KHz and 1 MHz. Point 59 on graph 58 is the point of full contact with vessel wall. After that point impedance rises slowly as stent is expanded. While the disclosure uses vascular stents as an example, the invention can also be used as a tool to measure the diameter of any lumen filled with a conductive liquid such as the urethra or blood vessel, even if a stent is not used. A long expanding balloon with multiple electrodes can be used to simultaneously measure a plurality of diameter in a vessel. One advantage of such a measuring tool than it has a very small diameter when the balloon is deflated.

An extra benefit from the invention is that it can sense the position of the stent relative to the balloon. This is important to detect any unintentional slip between the stent and balloon. Such slips are more common in stents which are crimped on the balloon at the point of use, such as at the hospital. If the stent slips even slightly relative to the balloon it will make contact with one of the sensing electrodes, greatly reducing the impedance to ground as the effective electrode area is greatly increased. This abnormal condition is easily detected by the sensing circuit without requiring any additional hardware in the balloon or in the detection circuits. By the way of example when the stent used for the tests of FIG. 6 was moved to touch one of the electrodes, the impedance to ground went down from 150 Ohm to 50 Ohm.

It is sometimes desirable to use one balloon for the initial deployment of the stent and a second balloon for a more precise expansion, or for expanding each end individually, as required in a tapered artery. In such cases it is important to sense the longitudinal alignment between the second balloon and the deployed stent. As the second balloon is moved into the deployed stent, the edge of the stent can be easily be detected using a single electrode. The impedance between the electrodes and the body drops sharply as soon as the electrode is inside the stent, even if it does not touch the stent. This is caused by the stent acting as a larger electrode, with lower impedance. Should the electrode touch the stent the impedance will drop even more. This drop is shown in FIG. 7. As long as the balloon is not inside the stent the impedance of either electrode to ground is fairly constant. As soon as one of the electrodes lines up with the edge of the stent there is a very sharp drop in impedance. This can be used to determine the longitudinal position to an accuracy of about 0.1 mm. This is of particular importance when two stents have to be placed next to each other to form a long stent or a bifurcated stent (Y-stent).

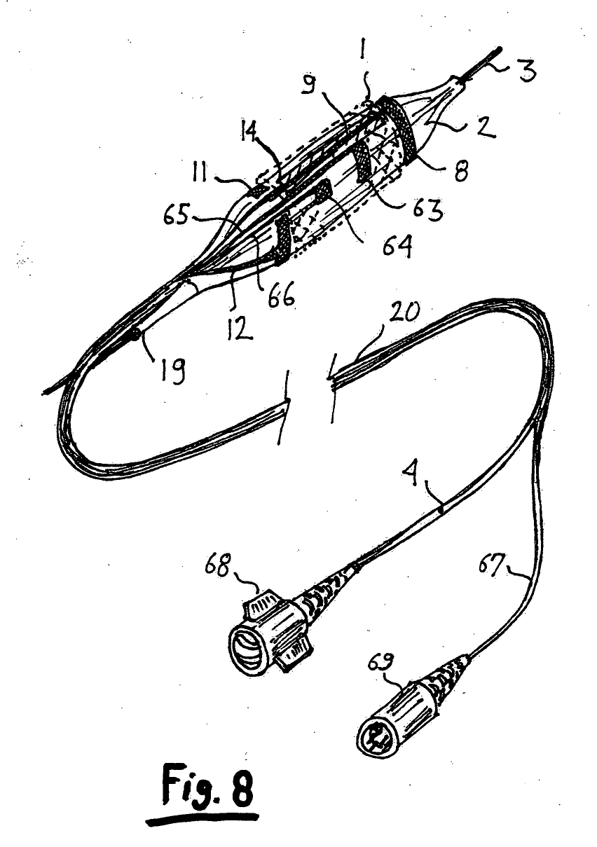

In some stent types, particularly Co—Cr stents, the stent tends to spring back to a smaller diameter when the pressure in the balloon is released. This effect can not be fully compensated by a calibration table supplied with the stent as the spring back is also dependent on tissue elasticity and on the amount the stent was expanded for a given pressure. The latter further depends on stiffness of the vessel. Since the balloon diameter changes in a predictable way with pressure, it is possible to sense the amount of spring back by slightly reducing balloon pressure until stent no longer is attached to balloon. At this point the balloon diameter is equal to the deployed stent diameter. The amount of pressure reduction required is approximately proportional to the spring back and provides guidance to the amount of over-pressurization required to compensate for the spring-back by further deforming the stent. For small amounts of spring back the process was found out to be linear: if spring back was equal to about 2 Atm of pressure, the amount of over-pressurizing needed to leave the stent at the nominal diameter was also 2 Atm. The point when the stent is no longer attached to the balloon will now be explained in conjunction with FIG. 8. The sensing can be done in one of three ways: without additional electrodes, with one electrode or with two or more electrodes. The preferred embodiment uses one electrode. To sense without additional electrodes, slight tension or compression is applied to pressurizing tube 4. As soon as the stent loses contact with the balloon the longitudinal slip will be detected as explained earlier. The balloon is re-positioned and re-pressurized to a higher pressure in order to further deform the stent. To sense with a single electrode, an additional conductive pad electrode 63, formed by metallizing balloon 2, is connected to sensing unit by metallized trace 65. All conductive traces on balloon 2 are connected by thin wires embedded in assembly 20 and wires 67 to electrical connector 69. Electrical connector 69 is used to terminate all electrical connections to balloon and form a connection to sensing unit shown in FIG. 5. As soon as the stent loses electrical contact with conductive pad 63 the impedance to ground increases as the effective electrode size decreases. As before, the pressure reduction associated with reaching this point is indicative of the spring-back of the stent. A third way of sensing this point is by simple conductive path sensing by two pads, 63 and 64, connected to connector 69 by traces 65 and 66. As soon as stent 2 loses contact with any one of the conductive pads 63 and 63, the impedance greatly increases as the conductivity of the blood is significantly less than that of the metallic stent. Fitting 68 is used to connect tube 4 to pressurizing device in the conventional manner. As explained earlier, only trace 9 needs to be covered by a thin insulating layer 14. All other traces, electrodes and conductive area can be left as a bare metallized coat which does not affect balloon dimensions of mechanical properties.

All sensing should be performed at low currents, in the range of uA to mA, to avoid any creation of gas bubbles by the hydrolysis of the blood. At very low currents the miniscule amounts of gas are easily dissolved in the blood.

It is possible at automate the complete stent placement sequence to include spring-back correction by using a pressurizing pump controlled by a computer as explained earlier. Computer controlled pumps are well known in the art. To control the complete sequence, the computer can follow these steps:
A. Pressurize balloon till full peripheral contact was reached by both the distal and proximal ends, as senses by the earlier describes method.
B. In case one end reaches contact full before the other, pressurize to a trade off pressure based on sensing the proximity of the other end to full contact. For example, if one end reaches 100% contact while the other indicates 80% contact, increase pressure till second end reads 95% contact or any other pre-programmed trade off.
C. Reduce pressure until stent loses electrical contact with spring-back detection electrode. Increase pressure above original pressure by an amount related to the pressure reduction needed to lose contact.
D. Reduce pressure, checking for new spring-back point. If stent diameter still too small repeat step C.

Clearly the same sequence can be followed by the cardiologist manually. The advantage of computerizing the sequence is that deployment time is reduced, thus reducing the period blood flow is blocked.

What is claimed is:

1. A method of expanding a stent inside a body lumen by using a stent balloon having at least one electrode outside the area covered by the stent, the method comprising of monitoring the electrical impedance between said electrode and the body, and determining the apposition of said stent inside said lumen by said impedance.

2. A method of expanding a stent inside a body lumen by using a stent balloon having at least one electrode outside the area covered by the stent, the method comprising of monitoring the electrical impedance between said electrode and the body, and determining the apposition of said stent inside said lumen by the rate of change of said impedance.

3. A method of positioning a stent balloon inside a body lumen relative to an existing stent, comprising:
adding at least one electrode to the balloon outside the area covered by the stent, and monitoring the electrical impedance between said electrode and the body.

4. A method as in claim 1 wherein each electrodes is made of a metallic ring deposited on the balloon and attached to a thin insulated electrical wire.

5. A method as in claim 2 wherein each electrodes is made of a metallic ring deposited on the balloon and attached to a thin insulated electrical wire.

6. A method as in claim 3 wherein each electrodes is made of a metallic ring deposited on the balloon and attached to a thin insulated electrical wire.

7. A method as in claim 1 wherein the electrical impedance is measured at multiple frequencies.

8. A method as in claim 3 wherein the electrical impedance is measured at multiple frequencies.

9. A method as in claim 1 wherein said balloon has two electrodes placed adjacent to the ends of said stent.

10. A method as in claim 2 wherein said balloon has two electrodes placed adjacent to the ends of said stent.

11. A method as in claim 3 wherein said balloon has two electrodes placed adjacent to the ends of said stent.

12. A method as in claim 1 wherein the electrical impedance is measured at a frequency of between 10 KHz and 10 MHz.

13. A method as in claim 1 wherein said balloon is removed after expanding the stent and a second balloon is inserted, said second balloon having at least one electrode used to locate said second balloon relative to stent and for stent apposition, based on monitoring electrical impedance between said electrode and body.

14. A method as in claim 3 wherein said stent balloon is used to accurately place a plurality of stents relative to each other.

15. A method as in claim 1 wherein said balloon is also used to measure stent spring-back.

16. A method as in claim 1 used for the automatic deployment of stents.

17. A method as in claim 2 used for the automatic deployment of stents.

18. A method as in claim 2 wherein a rapid increase in electrical impedance indicates correct stent apposition.
